(19) United States
(12) Patent Application Publication
(10) Pub. No.: US 2025/0304869 A1
(43) Pub. Date: Oct. 2, 2025

(54) METHOD FOR PRODUCING LIQUID FUEL

(71) Applicant: HONDA MOTOR CO., LTD., Tokyo (JP)

(72) Inventors: Kota Okamura, Wako-shi (JP); Shiori Nakajima, Wako-shi (JP); Akira Sonoda, Wako-shi (JP)

(21) Appl. No.: 19/089,067

(22) Filed: Mar. 25, 2025

(30) Foreign Application Priority Data
Mar. 28, 2024 (JP) 2024-053848

(51) Int. Cl. C10L 1/04; C07C 1/12

(52) U.S. Cl. CPC C10L 1/04; C07C 1/12

(57) ABSTRACT

What is provided is a method for producing a liquid fuel in which it is possible to increase the amount of carbon monoxide by supplying hydrogen and define a supply ratio between hydrogen and steam so that the H₂/CO ratio after gasification reaches two or higher. A method for producing a liquid fuel by which a liquid fuel is produced from a biomass raw material, the method having a gasification step of generating a synthesis gas from the biomass raw material, an electrolysis step of generating hydrogen from water with electric power generated using renewable energy, a liquid fuel production step of producing a liquid fuel using the synthesis gas generated by the gasification step and the hydrogen generated by the electrolysis step as raw materials, and a control step of controlling the gasification step and the electrolysis step, in which a mass of a biomass raw material (B), a mass of steam(S) and a mass of hydrogen (H) that are introduced into the liquid fuel production step satisfy the following formula (1) to formula (4).

$$H / B \leq - 0.031 \times S / B + 0.079 \qquad ( 1 )$$

$$H / B \geq - 0.028 \times S / B + 0.056 \qquad ( 2 )$$

$$S / B \geq 0.5 \qquad ( 3 )$$

$$H / B > 0 \qquad ( 4 )$$

### CROSS-REFERENCE TO RELATED APPLICATION

Priority is claimed on Japanese Patent Application No. 2024-053848, filed Mar. 28, 2024, the content of which is incorporated herein by reference.

### BACKGROUND OF THE INVENTION

### Field of the Invention

The present invention relates to a method for producing a liquid fuel.

### Description of Related Art

In recent years, as an alternative to fossil fuels, electrofuels for which hydrogen generated by electric power generated with renewable energy and a carbon source such as biomass or carbon dioxide that is discharged from factories are used as raw materials have been drawing attention.

A common procedure for producing a liquid fuel such as methanol or gasoline using biomass as a raw material is as described below. That is, a liquid fuel is produced from a biomass raw material by performing a gasification step of gasifying a biomass raw material that has undergone a predetermined pretreatment in a gasification furnace together with hydrogen, oxygen and steam to generate a synthesis gas containing hydrogen and carbon monoxide, a washing step of washing the generated synthesis gas to remove tar, a H₂/CO ratio adjustment step of adjusting the H₂/CO ratio of the synthesis gas that has undergone the washing step to a target ratio suitable for a liquid fuel to be produced, a desulfurization step of removing a sulfur component from the synthesis gas that has undergone the H₂/CO ratio adjustment step and a fuel production step of producing a liquid fuel from the synthesis gas that has undergone the desulfurization step.

### PATENT DOCUMENTS

[Patent Document 1] Japanese Unexamined Patent Application, First Publication No. 2021-147504

### SUMMARY OF THE INVENTION

In the gasification step, the biomass is thermally decomposed, and a gas mixture containing hydrogen and carbon monoxide, carbon dioxide, methane or the like is generated. In the gas that is generated by thermal decomposition, hydrogen and carbon monoxide serve as raw materials of the liquid fuel. In the case of making a liquid fuel composed of hydrogen and carbon monoxide, there is a need to control the index of the H₂/CO ratio to be an appropriate value. If it is possible to control the H₂/CO ratio to be within an appropriate range and operate the gasification furnace under conditions where the amount of carbon monoxide generated is maximized within the above-described H₂/CO ratio range, the yield of the liquid fuel can be maximized.

The present invention has been made in consideration of the above-described problem, and an object of the present invention is to provide a method for producing a liquid fuel in which it is possible to increase the amount of carbon monoxide by supplying hydrogen and define a supply ratio between hydrogen and steam so that the H₂/CO ratio after gasification reaches two or higher, which contributes to quality control improvement in production steps and, furthermore, increased energy efficiency.

In order to achieve the aforementioned object, the present invention provides the following means.

[1] A method for producing a liquid fuel by which a liquid fuel is produced from a biomass raw material, the method having:
a gasification step of generating a synthesis gas from the biomass raw material,
an electrolysis step of generating hydrogen from water with electric power generated using renewable energy,
a liquid fuel production step of producing a liquid fuel using the synthesis gas generated by the gasification step and the hydrogen generated by the electrolysis step as raw materials, and
a control step of controlling the gasification step and the electrolysis step,
in which a mass of a biomass raw material (B), a mass of steam(S) and a mass of hydrogen (H) that are introduced into the liquid fuel production step satisfy the following formula (1) to formula (4).

$$H / B \leq - 0.031 \times S / B + 0.079 \qquad ( 1 )$$

$$H / B \geq - 0.028 \times S / B + 0.056 \qquad ( 2 )$$

$$S / B \geq 0.5 \qquad ( 3 )$$

$$H / B > 0 \qquad ( 4 )$$

The present invention is capable of increasing the amount of carbon monoxide by supplying hydrogen and defining a supply ratio between hydrogen and steam so that the H₂/CO ratio after gasification reaches two or higher.

According to the present invention, it is possible to increase the amount of carbon monoxide by supplying hydrogen and define the supply ratio between hydrogen and steam so that the H₂/CO ratio after gasification reaches two or higher.

### BRIEF DESCRIPTION OF THE DRAWINGS

FIG. 1 is a view showing the configuration of a fuel production system that is used in a method for producing a liquid fuel according to one embodiment of the present invention.

FIG. 2 is a view showing investigation results of experiments of H₂/CO ratios and the amounts of liquid fuels generated.

FIG. 3 is a view showing investigation results of experiments of the H₂/CO ratios when S/B and H/B are changed.

FIG. 4 is a graph created based on FIG. 3 in which the vertical axis indicates H/B and the horizontal axis indicates S/B.

### DETAILED DESCRIPTION OF THE INVENTION

Hereinafter, a method for producing a liquid fuel according to one embodiment of the present invention will be described with reference to drawings.

### [Fuel Production System]

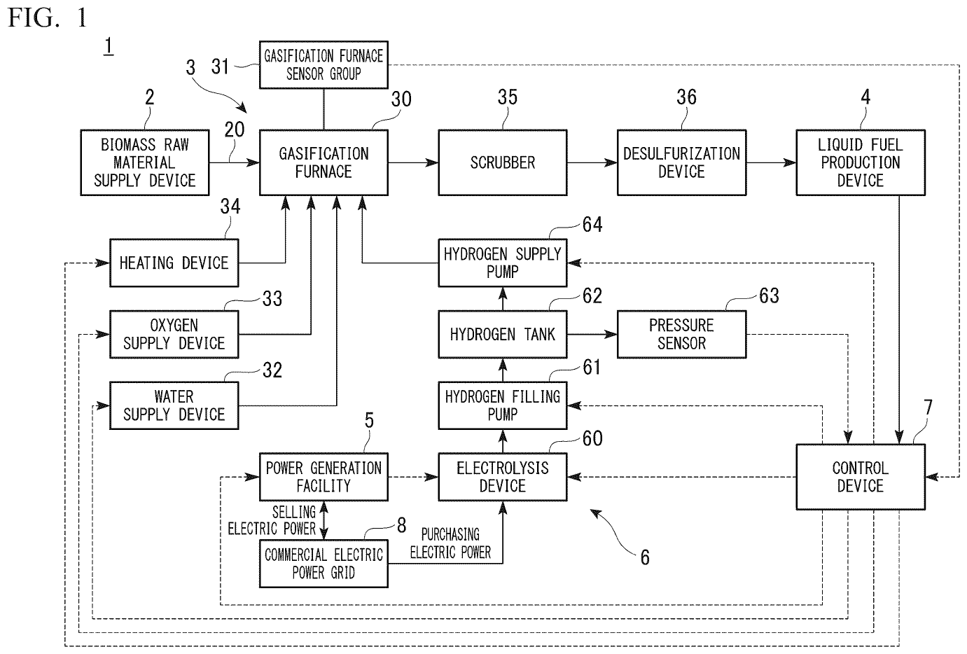

FIG. 1 is a view showing the configuration of a fuel production system according to one embodiment of the present invention.

As shown in FIG. 1, a fuel production system 1 includes a biomass raw material supply device 2 that supplies a biomass raw material, a gasification device 3 that gasifies the biomass raw material that is supplied from the biomass raw material supply device 2 and generates a synthesis gas containing hydrogen and carbon monoxide, a liquid fuel production device 4 that produces a liquid fuel using the synthesis gas that is supplied from the gasification device 3 and hydrogen generated with an electrolysis device 60 as raw materials, a power generation facility 5 that generates power using renewable energy, a hydrogen generation and supply device 6 that generates hydrogen and oxygen from water with electric power generated in the power generation facility 5 and supplies the generated hydrogen and oxygen to the gasification device 3 and a control device 7 that controls the gasification device 3, the power generation facility 5 and the hydrogen generation and supply device 6 and produces a liquid fuel from a biomass raw material with these devices.

The biomass raw material supply device 2 performs a predetermined pretreatment on the biomass raw material such as rice hulls, bagasse and wood and supplies the biomass raw material that has undergone this pretreatment to a gasification furnace 30 in the gasification device 3 through a raw material supply path 20. Here, examples of the pretreatment on the biomass raw material include a drying step of drying the raw material, a crushing step of crushing the raw material or the like.

The gasification device 3 includes the gasification furnace 30 that gasifies the biomass raw material that is supplied through the raw material supply path 20, a gasification furnace sensor group 31 that is composed of a plurality of sensors that detect the state of the inside of the gasification furnace 30, a water supply device 32 that supplies water into the gasification furnace 30, an oxygen supply device 33 that supplies oxygen or air into the gasification furnace 30, a heating device 34 that heats the gasification furnace 30, a scrubber 35 that washes a synthesis gas that is discharged from the gasification furnace 30 and a desulfurization device 36 that removes a sulfur component from the synthesis gas washed with the scrubber 35 and supplies the synthesis gas to the liquid fuel production device 4.

The water supply device 32 supplies water stored in a water tank, not shown, into the gasification furnace 30. The oxygen supply device 33 supplies oxygen stored in an oxygen tank, not shown, into the gasification furnace 30. The heating device 34 heats the gasification furnace 30 by consuming a fuel that is supplied from a fuel tank, not shown, or electric power that is supplied from a power supply, not shown. The amount of water supplied from the water supply device 32 into the gasification furnace 30, the amount of oxygen supplied from the oxygen supply device 33 into the gasification furnace 30 and the amount of heat injected from the heating device 34 into the gasification furnace 30 are controlled with the control device 7. In the fuel production system 1, there are cases where a necessity of proactively supplying water from the water supply device 32 into the gasification furnace 30 is removed by supplying hydrogen from the hydrogen generation and supply device 6, which will be described below, into the gasification furnace 30 or into the raw material supply path 20. In this case, it is also possible to exclude the water supply device 32 from the fuel production system 1.

When water, oxygen, heat and the like are injected into the gasification furnace 30 into which the biomass raw material has been injected with the water supply device 32, the oxygen supply device 33 and the heating device 34 described above, in the gasification furnace 30, a total of 10 kinds of gasification reactions as shown by, for example, the following formulae (1-1) to (1-5) and reverse reactions thereof progress, and a synthesis gas containing hydrogen and carbon monoxide is generated.

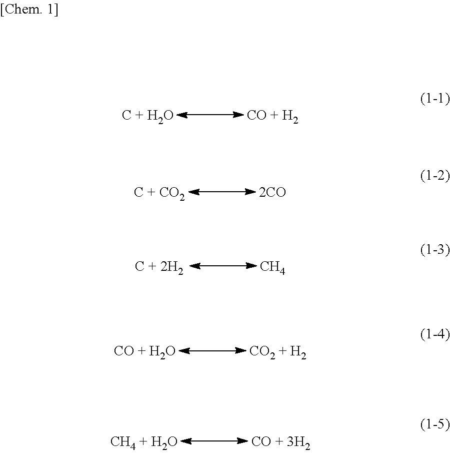

The gasification furnace sensor group 31 is composed of, for example, a pressure sensor that detects the pressure in the gasification furnace 30, a temperature sensor that detects the temperature in the gasification furnace 30, a H₂/CO sensor that detects the H₂/CO ratio corresponding the ratio between the hydrogen and the carbon monoxide in the synthesis gas in the gasification furnace 30, a CO₂ sensor that detects carbon dioxide in the gasification furnace 30 and the like. Detection signals of these sensors that configure the gasification furnace sensor group 31 are sent to the control device 7.

The gasification device 3 adjusts the H₂/CO ratio of the synthesis gas to a predetermined target ratio suitable for a liquid fuel intended to be produced (for example, the target ratio of the H₂/CO ratio is two in the case of producing methanol) by mixing hydrogen that is supplied from the hydrogen generation and supply device 6, which will be described below, with the synthesis gas that is generated by the gasification reactions shown by the formulae (1-1) to (1-5) and the reverse reactions thereof and then supplies this synthesis gas to the liquid fuel production device 4.

The liquid fuel production device 4 includes a methanol synthesis device, an MTG (Methanol To Gasoline) synthesis device, a FT (Fischer Tropsch) synthesis device, an upgrading device and the like and produces a liquid fuel such as methanol or gasoline from the synthesis gas having a predetermined H₂/CO ratio adjusted in the gasification device 3 using these devices.

The power generation facility 5 is composed of a wind power generation facility that generates power by wind, which is renewable energy, a solar power generation facility that generates power by sunlight, which is renewable energy, or the like. The power generation facility 5 is connected to the hydrogen generation and supply device 6, and electric power generated using renewable energy in the wind power generation facility, the solar power generation facility or the like can be supplied to the hydrogen generation and supply device 6. In addition, the power generation facility 5 is also connected to a commercial electric power grid 8. Therefore, it is also possible to supply a part or all of the electric power generated in the power generation facility 5 to the commercial electric power grid 8 and sell it to an electric power company.

The hydrogen generation and supply device 6 includes the electrolysis device 60, a hydrogen filling pump 61, a hydrogen tank 62, a pressure sensor 63 and a hydrogen supply pump 64, generates hydrogen by the electric power that is supplied from the power generation facility 5 using these devices and supplies the generated hydrogen to the gasification device 3.

The electrolysis device 60 is connected to the power generation facility 5 and generates hydrogen and oxygen from water through electrolysis by the electric power that is supplied from the power generation facility 5. In addition, the electrolysis device 60 is also connected to the commercial electric power grid 8. Therefore, the electrolysis device 60 is capable of generating hydrogen and oxygen not only by the electric power that is supplied from the power generation facility 5 but also by electric power that is purchased from an electric power company and supplied from the commercial electric power grid 8. The amount of hydrogen generated and the amount of oxygen generated by the electrolysis device 60 are controlled with the control device 7.

The hydrogen filling pump 61 compresses the hydrogen generated with the electrolysis device 60 and fills the inside of the hydrogen tank 62 with the hydrogen. The amount of the hydrogen filled with the hydrogen filling pump 61 is controlled with the control device 7. The hydrogen tank 62 stores the hydrogen compressed with the hydrogen filling pump 61. The pressure sensor 63 detects the tank internal pressure of the hydrogen tank 62 and sends a detection signal to the control device 7. The amount of hydrogen remaining in the hydrogen tank 62 is calculated with the control device 7 based on the detection signal of the pressure sensor 63. Therefore, in the present embodiment, remaining hydrogen amount acquisition means for acquiring the amount of hydrogen remaining in the hydrogen tank 62 is composed of the pressure sensor 63 and the control device 7.

The hydrogen supply pump 64 supplies the hydrogen stored in the hydrogen tank 62 to the gasification furnace 30 in the gasification device 3. The amount of the hydrogen supplied from the hydrogen supply pump 64 into the gasification furnace 30 is controlled with the control device 7. In FIG. 1, a case where the hydrogen stored in the hydrogen tank 62 is supplied into the gasification furnace 30 by the hydrogen supply pump 64 has been described, but the present invention is not limited thereto. The hydrogen stored in the hydrogen tank 62 may be supplied to the upstream side of the gasification furnace 30, more specifically, into the raw material supply path 20 of the biomass raw material.

The control device 7 is a computer that controls the amount of water supplied from the water supply device 32, the amount of oxygen supplied from the oxygen supply device 33, the amount of heat injected from the heating device 34, the amount of hydrogen generated with the electrolysis device 60, the amount of hydrogen filled with the hydrogen filling pump 61 and the amount of hydrogen supplied from the hydrogen supply pump 64 based on the detection signal from the gasification furnace sensor group 31, the detection signal from the pressure sensor 63 in the hydrogen tank 62 or the like.

### [Method for Producing Liquid Fuel]

The method for producing a liquid fuel according to one embodiment of the present invention is a method for producing a liquid fuel by which a liquid fuel is produced from a biomass raw material, the method having a gasification step of generating a synthesis gas from the biomass raw material, an electrolysis step of generating hydrogen from water with electric power generated using renewable energy, a liquid fuel production step of producing a liquid fuel using the synthesis gas generated by the gasification step and the hydrogen generated by the electrolysis step as raw materials and a control step of controlling the gasification step and the electrolysis step, in which the mass of a biomass raw material (B), the mass of steam(S) and the mass of hydrogen (H) that are introduced into the liquid fuel production step satisfy the following formula (1) to formula (4).

$$H / B \leq - 0.031 \times S / B + 0.079 \qquad ( 1 )$$

$$H / B \geq - 0.028 \times S / B + 0.056 \qquad ( 2 )$$

$$S / B \geq 0.5 \qquad ( 3 )$$

$$H / B > 0 \qquad ( 4 )$$

In the formula (1) to formula (4), H/B is the ratio of the mass of the hydrogen (H) to the mass of the biomass raw material (B), and S/B is the ratio of the mass of the steam(S) to the mass of the biomass raw material (B).

In a method for producing a liquid fuel using a gasification technique of a biomass raw material (Biomass to Liquid: BtL), the biomass raw material is thermally decomposed and converted into a gas, and a liquid fuel is then produced using hydrogen (H₂) and carbon monoxide (CO) that are contained in the gas. In the production of a liquid fuel by BtL, a reaction with any reaction formula of the following formula (5) to formula (7) is used. As shown in the following formula (5) to formula (7), an intended reaction does not occur if the H₂/CO ratio is not set to two.

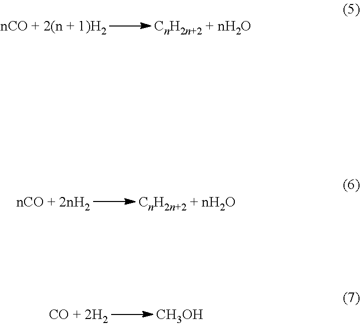

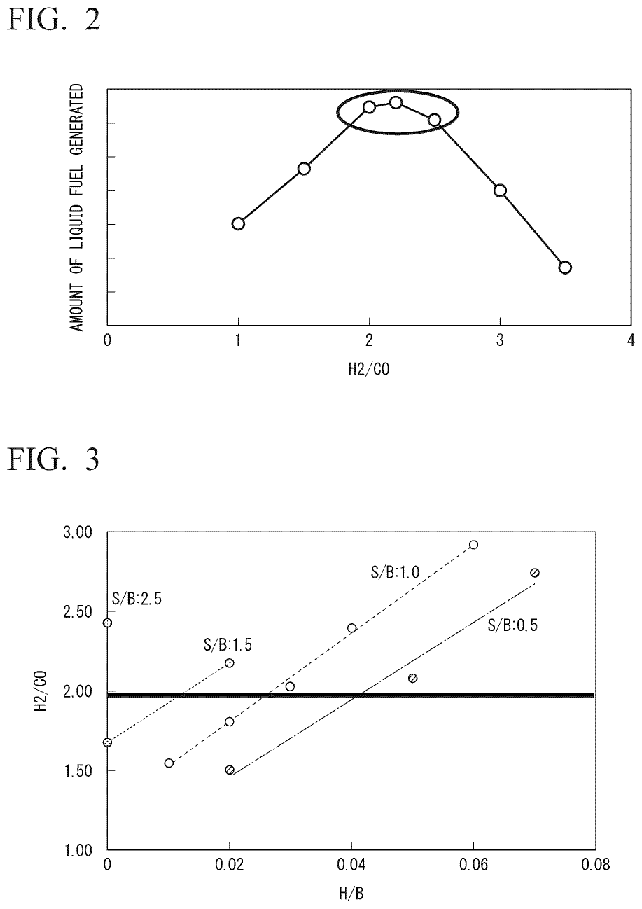

As a result of the present inventors' confirmation by test, it is found that, as shown in FIG. 2, when the H₂/CO ratio is 2.0 or higher and 2.5 or lower, the amount of the liquid fuel generated is maximized.

Therefore, the present inventors investigated, by experiments, the H₂/CO ratios when S/B and H/B were changed and consequently obtained results as shown in FIG. 3.

In FIG. 3, the amounts of carbon monoxide increased under all conditions when hydrogen was supplied. In FIG. 3, a region above the thick line in the graph is a region where the H₂/CO ratio reaches two or higher while the amount of carbon monoxide increases.

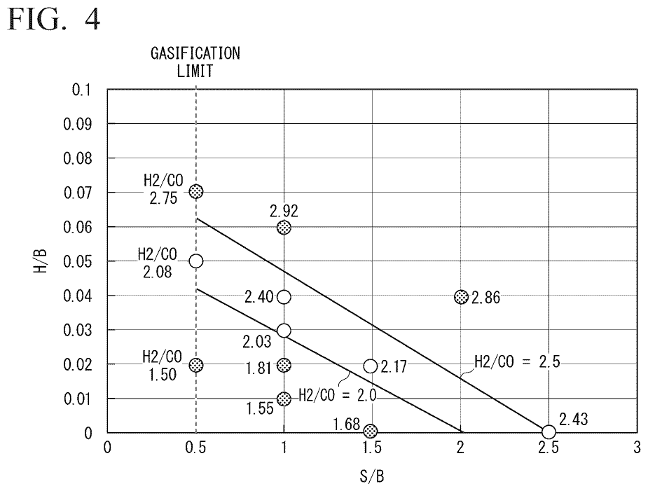

Based on FIG. 3, a graph shown in FIG. 4, in which the vertical axis indicates H/B and the horizontal axis indicates S/B, was created.

From the results shown in FIG. 4, ratios between hydrogen and steam at which ideal gasification was achieved were set from the experiment results.

A straight line for which the H₂/CO ratio is 2.0 is H/B=−0.028×S/B+0.056. In addition, a straight line for which the H₂/CO ratio is 2.5 is H/B=−0.031×S/B+0.079. Therefore, in order to set the H₂/CO ratio to 2.0 or higher and 2.5 or lower, there is a need to satisfy the formula (1) and the formula (2). That is, in the case of a region where H/B exceeds −0.031×S/B+0.079, since the H₂/CO ratio exceeds 2.5, the amount of a liquid fuel generated decreases. In addition, in the case of a region where H/B is less than −0.028×S/B+0.056, since the H₂/CO ratio reaches lower than 2.0, the amount of a liquid fuel generated decreases.

When the formula (3) is satisfied, it is possible to thermally decompose and gasify a biomass raw material.

When the formula (4) is satisfied, it is possible to produce a liquid fuel.

According to the method for producing a liquid fuel of the present embodiment, it is possible to increase the amount of carbon monoxide by supplying hydrogen and define the supply ratio between hydrogen and steam so that the H₂/CO ratio after gasification reaches two or higher.

Hitherto, the embodiment of the present invention has been described in detail, but the present invention is not limited to the embodiment and can be modified and changed in a variety of manner within the scope of the gist of the present invention described in the claims.

While preferred embodiments of the invention have been described and illustrated above, it should be understood that these are exemplary examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1 Fuel production system
2 Biomass raw material supply device
20 Raw material supply path
3 Gasification device
30 Gasification furnace
31 Gasification furnace sensor group
32 Water supply device
33 Oxygen supply device
34 Heating device
35 Scrubber
36 Desulfurization device
4 Liquid fuel production device
5 Power generation facility
6 Hydrogen generation and supply device
60 Electrolysis device
61 Hydrogen filling pump
62 Hydrogen tank
63 Pressure sensor
64 Hydrogen supply pump
65 Carbon dioxide collection device
66 Carbon dioxide tank
67 Carbon dioxide conversion device
68 Fuel synthesis device
7 Control device

What is claimed is:

1. A method for producing a liquid fuel by which a liquid fuel is produced from a biomass raw material, the method comprising:
a gasification step of generating a synthesis gas from the biomass raw material;
an electrolysis step of generating hydrogen from water with electric power generated using renewable energy;
a liquid fuel production step of producing a liquid fuel using the synthesis gas generated by the gasification step and the hydrogen generated by the electrolysis step as raw materials; and
a control step of controlling the gasification step and the electrolysis step,
wherein a mass of a biomass raw material (B), a mass of steam(S) and a mass of hydrogen (H) that are introduced into the liquid fuel production step satisfy formula (1) to formula (4):
$$H / B \leq - 0.031 \times S / B + 0.079 \qquad ( 1 )$$
$$H / B \geq - 0.028 \times S / B + 0.056 \qquad ( 2 )$$
$$S / B \geq 0.5 \qquad ( 3 )$$
$$H / B > 0. \qquad ( 4 )$$
